(19) United States
(12) Patent Application Publication
(10) Pub. No.: US 2025/0221883 A1
(43) Pub. Date: Jul. 10, 2025

(54) A BLOOD FLOW RESTRICTION GARMENT CONTROLLER, A BLOOD FLOW RESTRICTION GARMENT SYSTEM, A METHOD OF CONTROLLING A BLOOD FLOW RESTRICTION GARMENT, A METHOD OF CONTROLLING AN ELECTRONIC DEVICE AND COMPUTER SOFTWARE

(71) Applicant: SecondPerspective Ltd., Edinburgh (GB)

(72) Inventors: Alexander John Duncan BIRKS, Edinburgh (GB); Connor Michael MCGUILE, Glasgow (GB)

(73) Assignee: SecondPerspective Ltd., Edinburgh (GB)

(21) Appl. No.: 18/850,625

(22) PCT Filed: Mar. 22, 2023
(86) PCT No.: PCT/GB2023/050727

§ 371 Date: Sep. 25, 2024

(30) Foreign Application Priority Data
Mar. 25, 2022 (GB) 2204246.9

(51) Int. Cl. A61H 9/00

(52) U.S. Cl. CPC A61H 9/0078; A61H 2201/0103; A61H 2201/165; A61H 2201/5007; A61H 2201/5056; A61H 2201/5071; A61H 2205/06

(57) ABSTRACT

A blood flow restriction garment controller, a blood flow restriction garment system, a method of controlling a blood flow restriction garment, a method of controlling an electronic device and computer software. The blood flow restriction garment controller comprising: a pump arranged to increase a pressure in an air bladder of a blood flow restriction garment; an air pressure sensor arranged to measure a pressure of the air bladder and provide a signal indicative of the measured pressure; a valve arranged to control the pressure of the air bladder, wherein the valve has at least two operating states, wherein the operating states comprise an open state and a closed state; at least one or more of an inertial measurement unit, an accelerometer, a gyroscope, arranged to determine movement of the blood flow restriction garment controller and provide a signal indicative of the determined movement; processing means for providing a signal to control the valve by selecting one of the at least two operating states, wherein the signal to control the valve is based on at least one of the signal indicative of the measured pressure and the signal indicative of the determined movement.

This invention relates to blood flow restriction garment controller, a blood flow restriction garment system, a method of controlling a blood flow restriction garment, a method of controlling an electronic device and computer software.

### BACKGROUND

Blood Flow Restriction (BFR) training combines the partial occlusion of blood flow and low intensity exercise to mimic traditional high intensity exercise.

BFR training involves wrapping a compression garment around the top of a limb or a set of limbs and applying mechanical pressure to limit blood, and therefore oxygen, into a working group of muscles. The absolute mechanical pressure that should be applied varies from person to person based on limb circumference, tissue composition in the limb and other biological factors.

BFR is a scientifically validated method of improving musculature and the cardiovascular system in human subjects utilising low intensity exercise.

BFR training often requires manual adjustment of the compression garment by an operator, which can be time consuming. The compression garment often has a connection to an external pump via a connecting pipe, and a connection to an external controller which means the BFR training is often limited to a single room and is not portable.

### BRIEF SUMMARY OF THE DISCLOSURE

In accordance with the present invention there is provided a blood flow restriction garment controller comprising: a pump arranged to increase a pressure in an air bladder of a blood flow restriction garment; an air pressure sensor arranged to measure a pressure of the air bladder and provide a signal indicative of the measured pressure; a valve arranged to control the pressure of the air bladder, wherein the valve has at least two operating states, wherein the operating states comprise an open state and a closed state; at least one or more of an inertial measurement unit, an accelerometer, a gyroscope, arranged to determine movement of the blood flow restriction garment controller and provide a signal indicative of the determined movement; processing means for providing a signal to control the valve by selecting one of the at least two operating states, wherein the signal to control the valve is based on at least one of the signal indicative of the measured pressure and the signal indicative of the determined movement.

In some examples, the blood flow restriction garment controller comprises a communication module for communicating with an electronic device.

In some examples, the blood flow restriction garment controller is arranged to transmit at least one of the signal indicative of the measured pressure and the signal indicative of the determined movement to the electronic device using the communication module.

In some examples, the blood flow restriction garment controller comprises a housing arranged to removably attach to the blood flow restriction garment.

In some examples, the controller comprises a connector for the air pressure sensor to connect to the air bladder and a connector for the pump to connect to the air bladder, wherein the housing comprises one or more apertures to enable the connector for the air pressure sensor and the connector for the pump to connect to the air bladder.

In some examples, the blood flow restriction garment controller is arranged to be powered by portable energy storage means.

In some examples, the blood flow restriction garment controller is arranged to maintain the pressure in the air bladder.

In some examples, the processing means is arranged to control the pump in combination with controlling the valve to control the pressure in the air bladder.

In accordance with the present invention there is provided a blood flow restriction garment system, comprising:
the blood flow restriction garment controller of any of paragraphs to when the controller comprises a communication module for communicating with the electronic device;
the electronic device; wherein the electronic device comprises electronic device processing means, wherein the electronic device processing means are arranged to:
retrieve exercise data, wherein the exercise data indicates at least one exercise movement to be performed by a user of the blood flow restriction garment;
identify an exercise movement by the user based on analysis of at least one of the signal indicative of the measured pressure and the signal indicative of the determined movement using a machine learning model trained with an exercise movement data bank comprising movement data and pressure data associated with different types of exercise movement.

In some examples, the blood flow restriction garment system is arranged to:
control the pressure within the air bladder in dependence upon the identified exercise movement by the user and the exercise data;
send a control signal to the communication module of the blood flow restriction garment controller to control the valve to control the pressure in the air bladder.

In some examples, the exercise data indicates different levels of intensity for an exercise movement to be performed by the user, and wherein the electronic device determines that the pressure within the air bladder needs changing to enable the user to perform the exercise movement at the different levels of intensity.

In some examples, the exercise data targets maintaining the same mechanical work rate by the user for a number of repetitions of an exercise movement to be performed by the user, and wherein the electronic device determines that the pressure within the air bladder needs controlling to maintain the same mechanical work rate by the user for the repetitions.

In some examples, the exercise data targets maintaining the same mechanical work rate by the user for the period of time the exercise movement is to be performed by the user, and wherein the electronic device determines that the pressure within the air bladder needs controlling to maintain the same mechanical work rate by the user during the period of time.

In some examples, the exercise data indicates a sequence of exercise movements to be performed sequentially by the user, and wherein the electronic device determines that the pressure within the air bladder needs controlling for the next exercise movement in the sequence.

In some examples, the electronic device is arranged to determine that the identified exercise movement by the user deviates from the exercise data, and changes the exercise data in response to the deviation.

In some examples, the electronic device is arranged to determine that the identified exercise movement by the user deviates from the exercise data, and determines that the pressure within the air bladder needs controlling to reduce the deviation.

In some examples, the electronic device is arranged to change the exercise data based upon expert data and the identified exercise movement by the user.

In some examples, the electronic device is arranged to change the exercise data based upon the exercise movement data bank and the identified exercise movement by the user.

In some examples, the exercise data is based initially on user data, expert data and the exercise movement data bank.

In some examples, the system comprises the blood flow restriction garment.

In some examples, the blood flow restriction garment comprises a controller receiving portion arranged to receive the blood flow restriction garment controller to releasably attach the controller to the blood flow restriction garment.

In accordance with the present invention there is provided a method of controlling a blood flow restriction garment comprising:
receiving, at a processing means of a blood flow restriction garment controller, a signal indicative of a measured pressure of an air bladder of the blood flow restriction garment from an air pressure sensor of the blood flow restriction garment controller;
   receiving, at the processing means of the blood flow restriction garment controller, a signal indicative of a determined movement from at least one or more of an inertial measurement unit, an accelerometer, a gyroscope;
   providing a signal to control a valve of the blood flow restriction garment controller by selecting one of at least two operating states, the operating states comprising an open state and a closed state, wherein the signal to control the valve is based on at least one of the signal indicative of the measured pressure and the signal indicative of the determined movement.

In some examples, the method comprises transmitting at least one of the signal indicative of the measured pressure and the signal indicative of the determined movement to an electronic device using a communication module of the blood flow restriction garment controller.

In some examples, the method comprises maintaining the pressure in the air bladder.

In some examples, the method comprises controlling the pump in combination with controlling the valve to control the pressure in the air bladder.

In accordance with the present invention there is provided computer software, which, when executed on the processing means of the blood flow restriction garment

controller, performs the method of any of paragraphs [0027] to [0030]. Optionally the computer software is stored on a computer readable medium. Optionally the computer software is tangibly stored on a computer readable medium.

In a further aspect there is provided a non-transitory, computer-readable storage medium storing instructions thereon that, when executed by the processing means of the blood flow restriction garment controller, causes the processing means to carry out the method of any of paragraphs [0027] to [0030].

In accordance with the present invention there is provided a method of controlling an electronic device comprising:
retrieving exercise data, using electronic device processing means of the electronic device, wherein the exercise data indicates at least one exercise movement to be performed by a user of a blood flow restriction garment;
identifying exercise movement by the user, using the electronic device processing means, based on analysis of at least one of a signal indicative of a measured pressure of an air bladder of the blood flow restriction garment and a signal indicative of a determined movement using a machine learning model trained with an exercise movement data bank comprising movement data and pressure data associated with different types of exercise movement; wherein the signal indicative of the measured pressure of the air bladder and the signal indicative of the determined movement are received from a blood flow restriction garment controller.

In some examples, the method comprises:
controlling the pressure within the air bladder in dependence upon the identified exercise movement by the user and the exercise data;
sending a control signal to a communication module of the blood flow restriction garment controller to control the valve to control the pressure in the air bladder.

In some examples, the exercise data indicates different levels of intensity for an exercise movement to be performed by the user, and the method additionally comprises the electronic device determining that the pressure within the air bladder needs changing to enable the user to perform the exercise movement at the different levels of intensity.

In some examples, the exercise data targets maintaining the same mechanical work rate by the user for a number of repetitions of an exercise movement to be performed by the user, and the method additionally comprises the electronic device determining that the pressure within the air bladder needs controlling to maintain the same mechanical work rate by the user for the repetitions.

In some examples, the exercise data targets maintaining the same mechanical work rate by the user for the period of time the exercise movement is to be performed by the user, and the method additionally comprises the electronic device determining that the pressure within the air bladder needs controlling to maintain the same mechanical work rate by the user during the period of time.

In some examples, the exercise data indicates a sequence of exercise movements to be performed sequentially by the user, and the method additionally comprises the electronic device determining that the pressure within the air bladder needs controlling for the next exercise movement in the sequence.

In some examples, the method additionally comprises the electronic device determining that the identified exercise movement by the user deviates from the exercise data, and changing the exercise data in response to the deviation.

In some examples, the method additionally comprises the electronic device determining that the identified exercise movement by the user deviates from the exercise data, and determining that the pressure within the air bladder needs controlling to reduce the deviation.

In some examples, the method additionally comprises the electronic device changing the exercise data based upon expert data and the identified exercise movement by the user.

In some examples, the method additionally comprises the electronic device changing the exercise data based upon the exercise movement data bank and the identified exercise movement by the user.

In some examples, the method additionally comprises initially basing the exercise data on user data, expert data and the exercise movement data bank.

In accordance with the present invention there is provided computer software, which, when executed on the electronic device processing means, is arranged to perform the method of any of paragraphs [0033] to [0043]. Optionally the computer software is stored on a computer readable medium. Optionally the computer software is tangibly stored on a computer readable medium.

In a further aspect there is provided a non-transitory, computer-readable storage medium storing instructions thereon that, when executed by the electronic device processing means, causes the electronic device processing means to carry out the method of any of paragraphs [0033] to [0043].

In accordance with the present invention there is provided a method of controlling a blood flow restriction garment comprising:
receiving, at a processing means of a blood flow restriction garment controller, a signal indicative of a measured pressure of an air bladder of the blood flow restriction garment from an air pressure sensor of the blood flow restriction garment controller;
receiving, at the processing means of the blood flow restriction garment controller, a signal indicative of a determined movement from at least one or more of an inertial measurement unit, an accelerometer, a gyroscope;
providing a signal to control a valve of the blood flow restriction garment controller by selecting one of at least two operating states, the operating states comprising an open state and a closed state, wherein the signal to control the valve is based on at least one of the signal indicative of the measured pressure and the signal indicative of the determined movement;
retrieving exercise data, using electronic device processing means of the electronic device, wherein the exercise data indicates at least one exercise movement to be performed by a user of the blood flow restriction garment;
identifying exercise movement by the user, using the electronic device processing means, based on analysis of at least one of the signal indicative of a measured pressure of the air bladder of the blood flow restriction garment and the signal indicative of the determined movement using a machine learning model trained with an exercise movement data bank comprising movement data and pressure data associated with different types of exercise movement; wherein the signal indicative of the measured pressure of the air bladder and the signal indicative of the determined movement are received from the blood flow restriction garment controller.

In some examples, the method comprises the steps of any one of paragraphs [0028] to [0030] and [0034] to [0043].

In accordance with the present invention there is provided computer software, which, when executed on the processing means of the controller and the electronic device processing means, is arranged to perform any method disclosed herein. Optionally the computer software is stored on a computer readable medium. Optionally the computer software is tangibly stored on a computer readable medium.

In a further aspect there is provided a non-transitory, computer-readable storage medium storing instructions thereon that, when executed by the processing means of the controller and the electronic device processing means, causes the processing means of the controller and the electronic device processing means to carry out any method disclosed herein.

In accordance with the present invention there is provided an electronic device comprising electronic device processing means, wherein the electronic device processing means are arranged to:
retrieve exercise data, wherein the exercise data indicates at least one exercise movement to be performed by a user of a blood flow restriction garment;
identify an exercise movement by the user based on analysis of at least one of the signal indicative of a measured pressure and a signal indicative of the determined movement using a machine learning model trained with an exercise movement data bank comprising movement data and pressure data associated with different types of exercise movement, wherein the signal indicative of a measured pressure and the signal indicative of the determined movement are received from a blood flow restriction garment controller.

In some examples, the electronic device is arranged to:
control the pressure within the air bladder in dependence upon the identified exercise movement by the user and the exercise data;
send a control signal to the communication module of the blood flow restriction garment controller to control the valve to control the pressure in the air bladder.

In some examples, the exercise data indicates different levels of intensity for an exercise movement to be performed by the user, and wherein the electronic device determines that the pressure within the air bladder needs changing to enable the user to perform the exercise movement at the different levels of intensity.

In some examples, the exercise data targets maintaining the same mechanical work rate by the user for a number of repetitions of an exercise movement to be performed by the user, and wherein the electronic device determines that the pressure within the air bladder needs controlling to maintain the same mechanical work rate by the user for the repetitions.

In some examples, the exercise data targets maintaining the same mechanical work rate by the user for the period of time the exercise movement is to be performed by the user, and wherein the electronic device determines that the pressure within the air bladder needs controlling to maintain the same mechanical work rate by the user during the period of time.

In some examples, the exercise data indicates a sequence of exercise movements to be performed sequentially by the user, and wherein the electronic device determines that the pressure within the air bladder needs controlling for the next exercise movement in the sequence.

In some examples, the electronic device is arranged to determine that the identified exercise movement by the user deviates from the exercise data, and changes the exercise data in response to the deviation.

In some examples, the electronic device is arranged to determine that the identified exercise movement by the user deviates from the exercise data, and determines that the pressure within the air bladder needs controlling to reduce the deviation.

In some examples, the electronic device is arranged to change the exercise data based upon expert data and the identified exercise movement by the user.

In some examples, the electronic device is arranged to change the exercise data based upon the exercise movement data bank and the identified exercise movement by the user.

In some examples, the exercise data is based initially on user data, expert data and the exercise movement data bank.

### BRIEF DESCRIPTION OF THE DRAWINGS

FIG. 1A illustrates an example blood flow restriction garment controller.

FIG. 1B illustrates an example processing means of an example blood flow restriction garment controller.

FIG. 2 illustrates an example blood flow restriction garment controller and an electronic device.

FIG. 3A and 3B illustrate an example blood flow restriction garment controller and an example blood flow restriction garment.

FIG. 4 illustrates an example blood flow restriction garment controller.

FIG. 5A illustrates an example blood flow restriction garment controller in cross section.

FIG. 5B illustrates an example blood flow restriction garment controller and blood flow restriction garment in cross section.

FIG. 6 illustrates an example schematic.

FIGS. 7A and 7B illustrate example schematics.

FIG. 8A and 8B illustrate an example blood flow restriction garment system

FIG. 9 illustrates example exercise data

FIG. 10 illustrates example exercise data and data inputs

FIG. 11 illustrates a machine learning model.

FIGS. 12A and 1B illustrate a user and an example blood flow restriction garment controller and an example blood flow restriction garment.

FIG. 13 illustrates a method.

FIG. 14 illustrates a method

### DETAILED DESCRIPTION

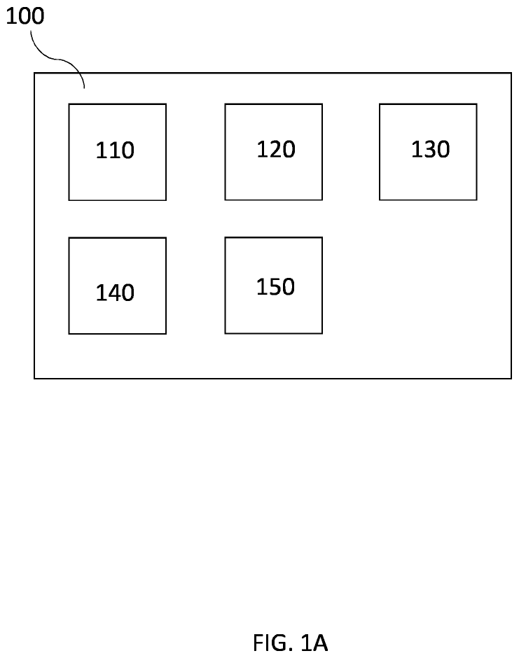

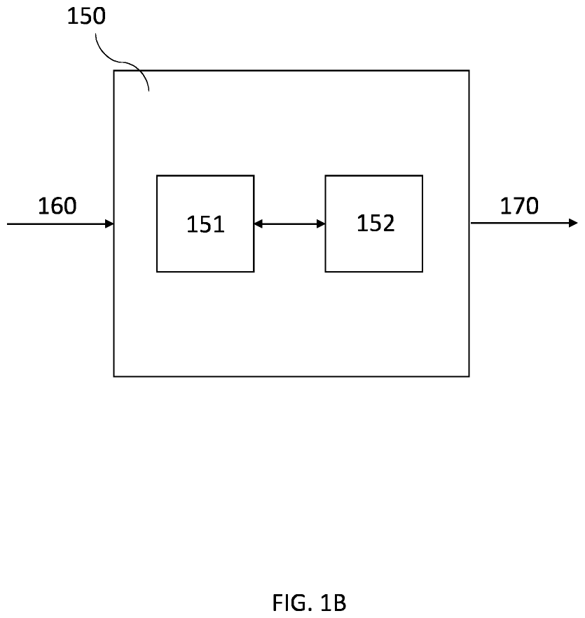

FIG. 1A illustrates an example blood flow restriction garment controller 100. The blood flow restriction garment controller 100 comprises a pump 110 arranged to increase a pressure in an air bladder of a blood flow restriction garment.

The controller 100 also comprises an air pressure sensor 120 arranged to measure the pressure of the air bladder and provide a signal indicative of the measured pressure.

The controller 100 also comprises a valve 130 arranged to control the pressure of the air bladder. The valve 130 has at least two operating states comprising an open state and a closed state.

The controller 100 also comprises at least one or more of an inertial measurement unit 140, an accelerometer 141, a gyroscope 142. In the example of FIG. 1A, the controller 100 comprises an inertial measurement unit 140. The at least one or more of an inertial measurement unit 140, an accelerometer 141, a gyroscope 142 are arranged to determine movement of the blood flow restriction garment controller 100 and provide a signal indicative of the determined movement.

The controller 100 also comprises processing means 150 for providing a signal to control the valve 130 by selecting one of the at least two operating states, wherein the signal to control the valve 130 is based on at least one of the signal indicative of the measured pressure and the signal indicative of the determined movement.

The valve 130 can be, for example, a solenoid valve.

The controller 100 illustrated in FIG. 1A provides the advantage that the blood flow restriction garment can be automatically controlled to change the pressure in the air bladder in dependence upon the movement of the user, which is detected via determining that the controller 100 has moved. It also provides the advantage that the measured pressure can be used to automatically control the pressure in the air bladder.

FIG. 1B illustrates an example processing means 150 of an example blood flow restriction garment controller 100. The processing means 150 in this example comprises a processor 151 and a memory 152. The processor 151 is arranged to operably execute computer software/computer program code thereon, where the computer software/computer program code is stored in a computer-readable medium accessible to the processor 151. The computer-readable medium may be one or more memory devices, where the one or more memory devices may also store data for use by the software/program code (e.g. memory 152 or a separate memory store external to the processing means 150).

The processing means 150 can receive data as one or more inputs 160 and can provide data as one or more outputs 170. It will be appreciated that in other examples that the memory 152 is not part of the processing means 150, but is part of a physically distinct computer system accessible to the processing means 150. In some examples, the processing means 150 can be referred to as a processing unit 150.

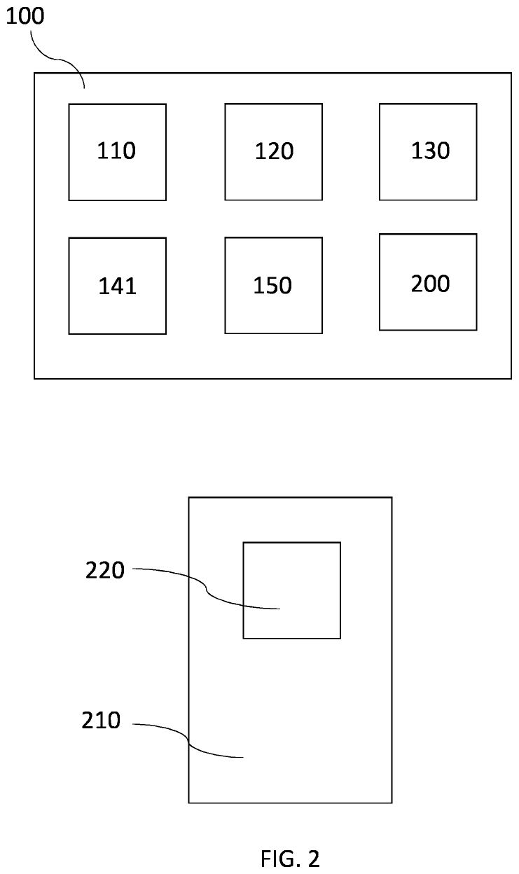

FIG. 2 illustrates an example blood flow restriction garment controller 100. The controller 100 comprises a communication module 200 for communicating with a communication module 220 of an electronic device 210. The communication module 200 of the controller 100 can comprise a Bluetooth module, which communicates with the communication module 220 of the electronic device 210. The communication module 220 of the electronic device 210 can comprise a Bluetooth module. In other examples the communication module 200 of the controller 100 and the communication module 220 of the electronic device 210 can be connected via a computer network comprising one or more networks, such as a Local Area Networks (LANs), and the internet.

The blood flow restriction garment controller 100 is arranged to transmit at least one of the signal indicative of the measured pressure and the signal indicative of the determined movement to the electronic device 210 using the communication module 200 of the controller 100.

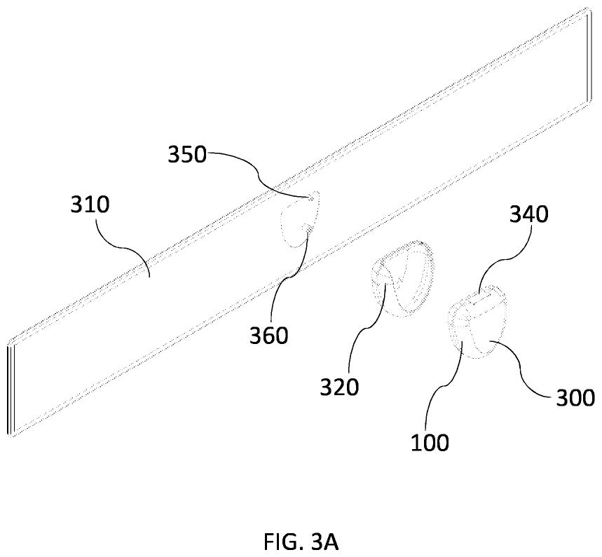

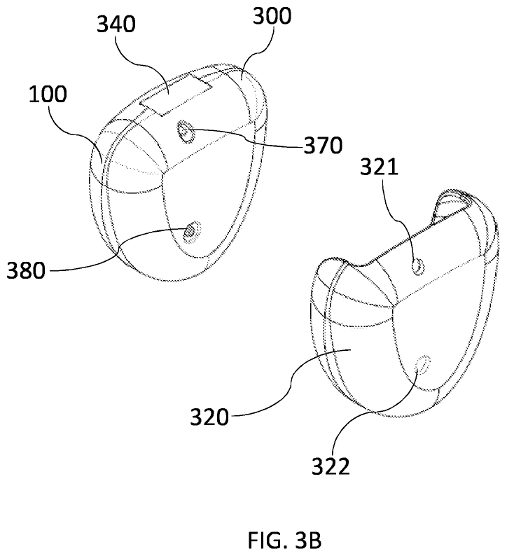

FIG. 3A illustrates an example blood flow restriction garment controller 100. In this example, the controller 100 comprises a housing 300 arranged to removably attach to a blood flow restriction garment 310.

In this example, the blood flow restriction garment 310 comprises a controller receiving portion 320, which can be referred to as a holder or a bumper. The receiving portion 320 is arranged so that the controller 100 releasably snap-fits into the receiving portion 320. In this example, the receiving portion 320 comprises a deformable hard plastic, so that the sidewalls of the receiving portion 320 can deform slightly to allow the housing 300 to snap-fit into the receiving portion 320. The controller 100 can be released from the receiving portion 320 by pushing the controller to slightly deform the sidewalls of the receiving portion 320.

In the example of FIG. 3A, the receiving portion 320 of the blood flow restriction garment 310 is shown as being separated from the rest of the garment 310. The receiving portion 320 can be releasably attached to the rest of garment 310. In other examples, the receiving portion 320 can be welded or adhered to the rest of the garment 310, and/or can be sewn into fabric of the garment 310.

In the example of FIG. 3A, the controller 100 comprises a screen 340, which is discussed below.

In the example of FIG. 3A, the garment 310 comprises a first air bladder connector 350 and a second air bladder connector 360.

As illustrated in FIG. 3B, the controller 100 in this example comprises a connector 380 for the air pressure sensor 120 to connect to the air bladder and a connector 370 for the pump 110 to connect to the air bladder. The connector 370 for the pump 110 to connect to the air bladder can connect to the first air bladder connector 350 and the connector 380 for the air pressure sensor 120 to connect to the air bladder can connect to the second air bladder connector 360.

The connector 370 for the pump 110, the connector 380 for the air pressure sensor 120, the first air bladder connector 350, the second air bladder connector 360 can comprise flexible tubing to enable a friction seal when mating the connector 370 for the pump 110 to the first air bladder connector 350 and the connector 380 for the air pressure sensor to the second air bladder connector 360. For example, the diameter of the flexible tubing of the connector 370 for the pump 110 and the connector 380 for the air pressure sensor 120 can be sized so that it can fit within the flexible tubing of the first air bladder connector 350 and the second air bladder connector 360 respectively by contacting the interior surface of the first air bladder connector 350 and second air bladder connector 360 causing it to flex slightly to accommodate the connector 370 for the pump 110 and the connector 380 for the air pressure sensor 120.

The frictional contact between the connector 370 for the pump 110 and the first air bladder connector 350 and the connector 380 for the air pressure sensor 120 and the second air bladder connector 360 seals the connection between the pump 110 and the air bladder and the air pressure sensor 120 and the air bladder.

As illustrated in FIG. 3B, the housing 300 of the controller 100 has openings for the connector 370 for the pump 110 and the connector 380 for the air pressure sensor 120. The receiving portion 320 comprises openings 321, 322 to allow the connector 370 for the pump 110 and the connector 380 for the air pressure sensor 120 to pass through to enable connection to the first air bladder connector 350 and the second air bladder connector 360.

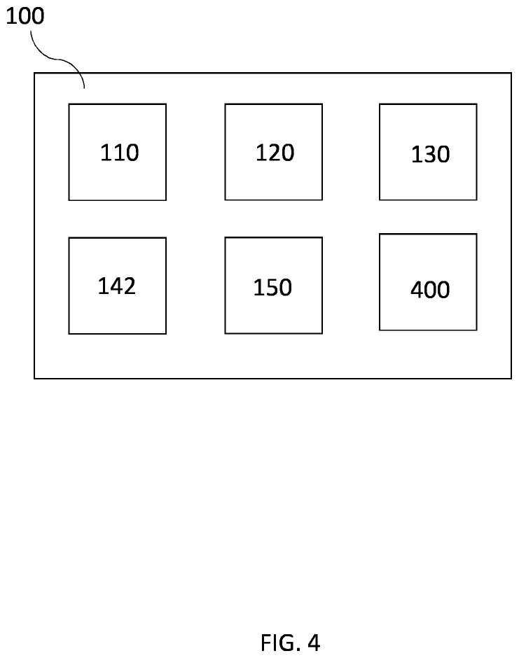

FIG. 4 illustrates an example blood restriction garment controller 100. In this example, the blood flow restriction garment controller 100 is arranged to be powered by portable energy storage means 400. In this particular example, the controller 100 comprises a portable energy storage means 400. The portable energy storage means 400 can be one or more rechargeable batteries, for example one or more Li-ion batteries that is integrated into the housing 300 and is rechargeable via a cable and a connection built into the controller 100. In other examples, the portable energy storage means 400 can comprise one or more removable batteries, and the housing 300 can comprise an openable battery compartment and battery terminals which enables easy replacement of the one or more batteries.

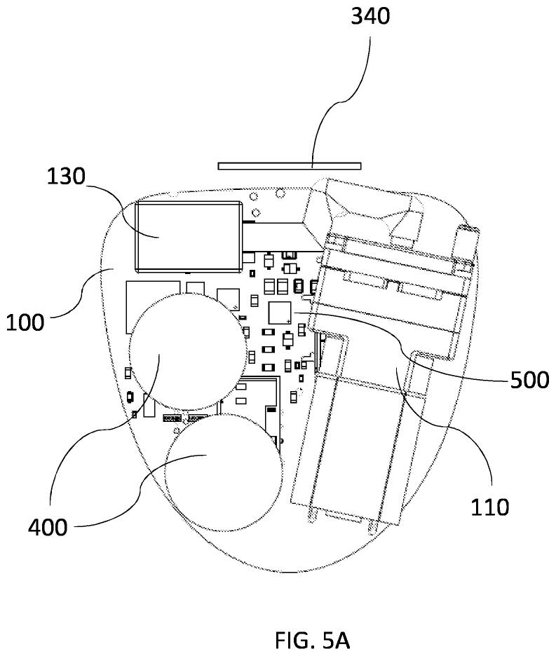

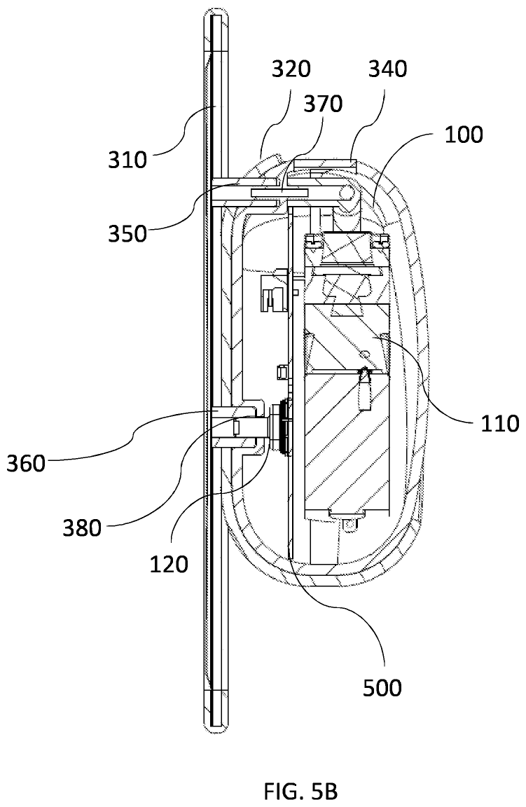

FIG. 5A illustrates an example blood restriction garment controller 100 in cross-section. In this example the portable energy storage means 400 comprises two batteries. The controller 100 in this example comprises a printed circuit board 500. The processing means 150, the at least one or more of an inertial measurement unit 140, an accelerometer 141, a gyroscope 142, are provided on the printed circuit board 500. The air pressure sensor 120 is not visible in FIG. 5A.

FIG. 5B illustrates an example blood restriction garment controller 100 in cross-section, where the cross-section is along an axis perpendicular to that shown in FIG. 5A. FIG. 5B illustrates the controller 100 attached to the blood flow restriction garment 310. FIG. 5B also illustrates the connector 370 for the pump 110 connected to the first air bladder connector 350 and the connector 380 for the air pressure sensor 120 connected to the second air bladder connector 360. The air pressure sensor 120 is shown, connected to the connector 380.

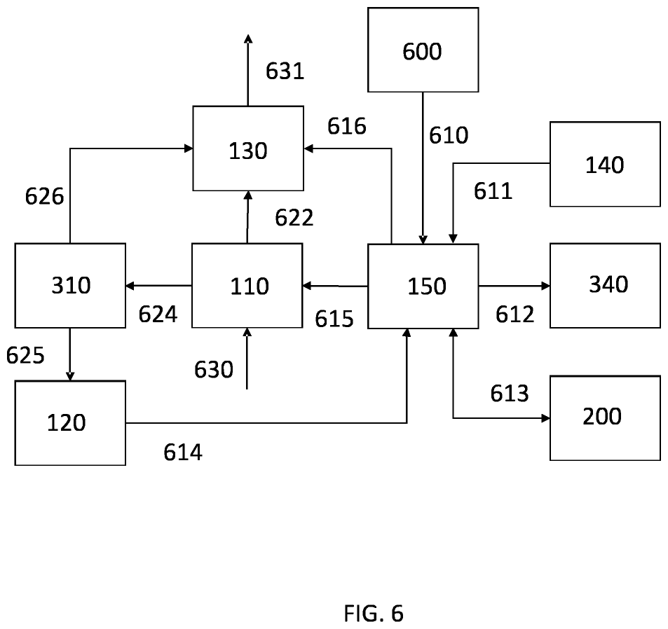

FIG. 6 illustrates an example schematic of signals and air connections between components of an example blood flow restriction garment controller 100 and the blood flow restriction garment 310. The air connections can comprise tubing. In this example, the controller 100 comprises at least one user control 600, such as at least one button. One or more of the at least one user control 600 is arranged to provide a signal 610 as an input to the processing means 150 to cause the controller 100 to turn on or turn the controller 100 off.

The processing means 150 can receive a signal 614 as an input from the air pressure sensor 120 which is indicative of a pressure measured by the air pressure sensor 120, via air connection 625 between the air bladder of the blood flow restriction garment 310 and the air pressure sensor 120. The processing means 150 can provide a signal 616 as an output to the valve 130 to control the valve 130 by selecting one of the at least two operating states, in order to control the pressure in the air bladder of the blood flow restriction garment 310.

For example, if the valve 130 was previously in the closed state and the signal 616 controls the valve to change to the open state, the pressure in the air bladder can decrease via air connection 626 between the blood flow restriction garment 310 and the valve 130 and out through a valve exit 631 to the atmosphere.

The signal 616 to control the valve 130 is based on at least one of the signal 614 indicative of the measured pressure and the signal 611 indicative of a determined movement from the inertial movement sensor 140.

In an example, the signal 614 from the air pressure sensor 120 indicates a pressure which exceeds a maximum allowed pressure, which can be stored on memory 152, or located in a memory accessible to the controller 100, for example via communication module 200. In response to this, the processing means 150 can send the signal 616 to control the valve to change to the open state to allow the pressure in the air bladder of the blood flow restriction garment 310 to reduce so that it does not exceed the maximum allowed pressure. The processing means 150 could also send a signal 615 to the pump to turn off to help to reduce the pressure in the air bladder.

In an example, the controller 100 can send data indicative of the measured pressure and/or data indicative of the determined movement to an external device, such as electronic device 210, using communication module 200 of the controller 100, via a signal 613. The external device, such as electronic device 210, can then process the data indicative of the measured pressure and/or indicative of the determined movement and can then send data to the controller 100, via a signal 613 from the communication module 200 of the controller 100 to the controller 100 which causes the controller 100 to send a signal 616 to control the valve 130 to control the pressure in the air bladder of the blood flow restriction garment 310.

In some examples, the external device, such as the electronic device 210, can communicate with one or more other external devices to process/analyse the data indicative of the measured pressure and/or the data indicative of the determined movement.

In an example, the processing means 150 is arranged to control the pressure in the air bladder of the blood flow restriction garment 310 by maintaining the pressure at a certain value. To maintain the pressure at the certain value the processing means 150 can control valve 130 and/or the pump 110 to increase or decrease the pressure in the air bladder, using the signal 614 indicative of the measured pressure to determine whether the pressure needs increasing or decreasing to maintain the pressure at the certain value.

In an example, the processing means 150 is arranged to control the valve 130 and/or the pump 110 to control the pressure in the air bladder. For example, the processing means 150 can control the pump 110 in combination with the valve 130 to control the pressure in the air bladder. For example, the processing means 150 could determine or be instructed to increase the pressure in the air bladder and maintain it at a first value. To accomplish this, the processing means 150 can turn the pump 110 on if it isn't already and control the valve 130 to be in the closed state, thereby preventing air leaving the air bladder to the atmosphere. Because air cannot escape to the atmosphere, the pump 110 causes the pressure in the air bladder to increase. When the air pressure sensor 120 sends a signal 614 indicative of a measured pressure that matches the first value, or within a tolerated difference, the processing means 150 can turn the pump off. The processing means 150 can then maintain the pressure at the first value as described previously, until the processing means 150 determines or is instructed that the pressure in the air bladder needs to change.

In an example, the processing means 150 could determine or be instructed that the pressure in the air bladder needs increasing to a second value, and can control the pump 110 to turn on and the valve 130 to be in the closed state to cause the increase in pressure to the second value.

In an example, the processing means 150 could determine or be instructed that the air bladder needs to be depressurised, and control the pump 110 to be turned off and to control the valve 130 to be in the open state.

In the example of FIG. 6, pump 110 has an air connection 624 to the blood flow restriction garment 310 so that the pump 110 can increase the pressure in the air bladder of the blood flow restriction garment 310. The pump has an air intake 630 which takes air in from the atmosphere. The pump 110 also has an air connection 622 to the valve 130. This means that air exiting the pump 110 can exit the valve 130 and into the atmosphere via valve exit 631 if the valve is in the open state.

In an example, the controller can send data via signal 612 to the screen 340, which is described below.

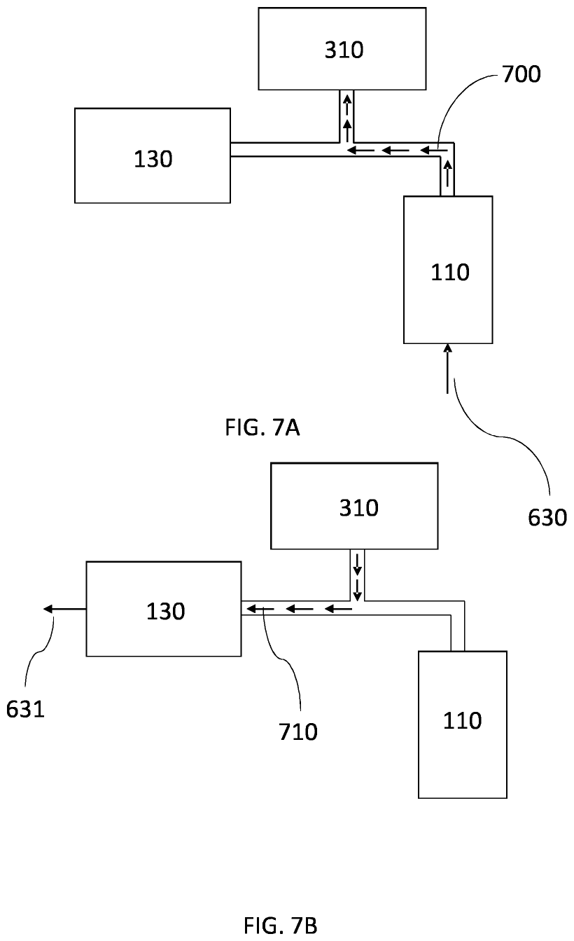

FIG. 7A and FIG. 7B illustrate an example pump 110, valve 130 and blood flow restriction garment 310. In FIG. 7A the valve 130 is in the closed state and the arrows on path 700 illustrate air from the pump 110 entering the air bladder of the blood flow restriction garment 310 to increase the pressure in the air bladder.

In FIG. 7B, the valve 130 is in the open state and the pump 110 is not providing air to the blood flow restriction garment 310 because, for example, the pump 110 is turned off. The path 710 represented by the arrows illustrates air leaving the blood flow restriction garment 310 and exiting to the atmosphere via valve exit 631, causing pressure in the air bladder of the blood flow restriction garment 310 to decrease. The pump 110 can comprise a one-way pump valve, so that air cannot re-enter the pump 110 whilst it is not providing air to the blood flow restriction garment 310.

In some examples, the valve 130 is arranged to have other operating states besides the open state and the closed state. For example, the valve 130 can have at least one partially open state, where the valve is not as open as it is in the open state, so that the rate of decrease in pressure of the air bladder of the blood flow restriction garment 310 can be lower compared to the rate of decrease in pressure whilst the valve is in the open state.

In an example, the pump 110 is arranged to provide air to the blood flow restriction garment 310 whilst the valve 130 is in the open state or in one of the more of the at least one partially open states, which provides another way to control the pressure in the air bladder of the blood flow restriction garment 310.

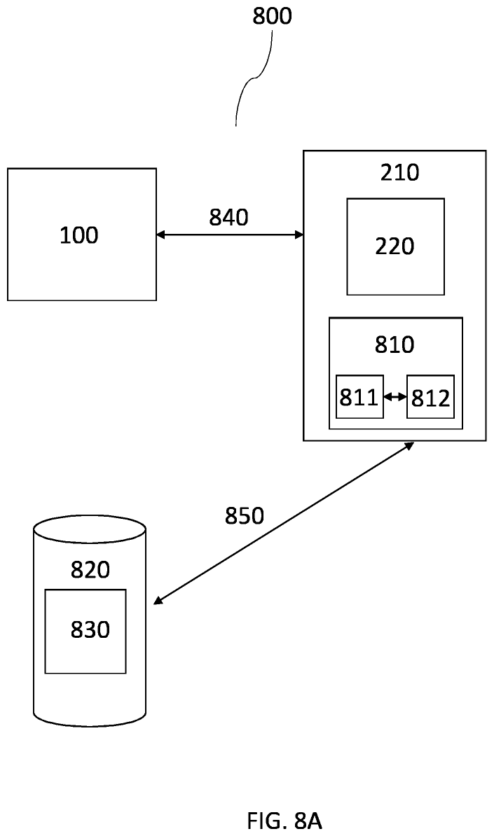

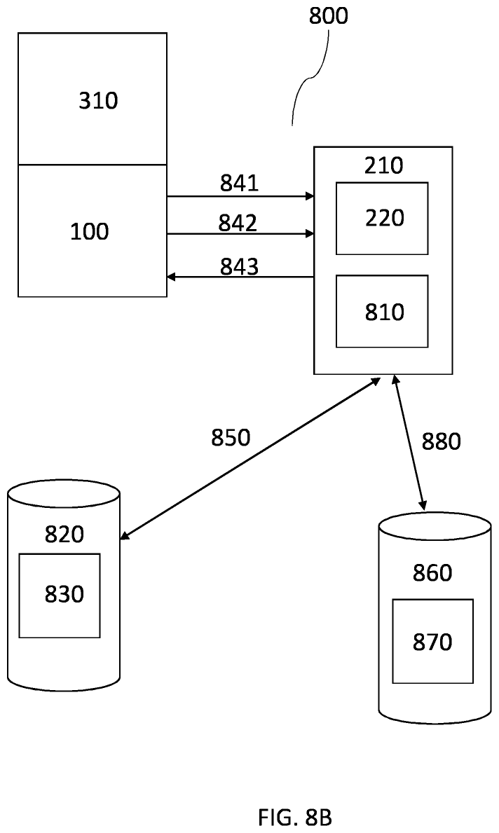

FIG. 8A illustrates an example blood flow restriction garment system 800. The system 800 comprises the blood flow restriction garment controller 100 of any of the previously described examples. It also comprises the electronic device 210 as previously described. The electronic device 210 comprises electronic device processing means 810. In this example, the electronic device processing means 810 comprises a processor 811 and a memory 812. The processor 811 is arranged to operably execute computer software/computer program code thereon, where the computer software/computer program code is stored in a computer-readable medium accessible to the processor 811. The computer-readable medium may be one or more memory devices, where the one or more memory devices may also store data for use by the software/program code (e.g. memory 812 or a separate memory store external to the electronic device processing means 910).

The electronic device processing means 810 can receive data as one or more inputs and can provide data as one or more outputs. It will be appreciated that in other examples that the memory 812 is not part of the electronic device processing means 810, but is part of a physically distinct computer system accessible to the electronic device processing means 810. In some examples, the electronic device processing means 810 can be referred to as an electronic device processing unit 810.

The electronic device processing means 810 can retrieve exercise data. The exercise data indicates at least one exercise movement to be performed by a user of the blood flow restriction garment 310. In this example, the electronic device processing means 810 has the exercise data stored in the memory 812.

The electronic device processing means 810 can also identify an exercise movement by the user based on analysis of at least one of the signal indicative of the measured pressure and the signal indicative of the determined movement using a machine learning model 830 trained with an exercise movement data bank. In this example, the machine learning model is stored in a data store 820 separate to the electronic device 210, for example in a server.

The controller 100 and electronic device 210 communicate via communication link 840 using the communication module 200 of the controller 100 and the communication module 220 of the electronic device 210. The electronic device 210 and the data store 820 communicate via communication link 850 using the communication module 220.

FIG. 8B illustrates an example blood flow restriction garment system 800. In this example, the system 800 comprises the blood flow restriction garment 310, which is attached to the controller 100.

In the example of FIG. 8B, the exercise data 870 is stored in a data store 860, and is retrievable by the electronic device processing means 810 via a communication link 880 using the communication module 220 of the electronic device 210.

FIG. 8B illustrates signals that can be sent by the communication link 840. A signal 841 containing data indicative of the measured pressure is sent from the controller 100 to the electronic device 210 and a signal 842 containing data indicative of the determined movement is sent from the controller 100 to the electronic device 210.

In this example, the system 800 is arranged control the pressure within the air bladder in dependence upon the identified exercise movement by the user and the exercise data. To control the pressure within the air bladder, the electronic device 210 can send a control signal 843 to the communication module 200 of the blood flow restriction garment controller 100 to control the valve 130, so as to control the pressure in the air bladder.

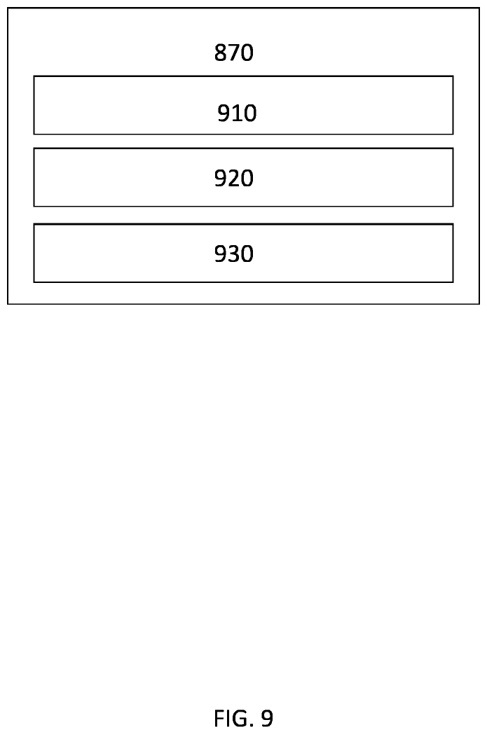

FIG. 9 illustrates example exercise data 870. The exercise data 870 can comprise one or more exercise instructions 910, 920, 930. The one or more exercise instructions 910, 920, 930 indicate an exercise movement that is to be performed by a user, and may also comprise exercise conditions.

An example exercise instruction indicates different levels of intensity of an exercise movement to be performed by the user as an exercise condition. When the exercise data 870 is retrieved by the electronic device processing means 810, the electronic device processing means 810 determines or is instructed that the exercise movement to be performed and the intensity level required corresponds to a target pressure value or multiple target pressure values during the time period the exercise movement is performed for the garment 310 and sends a control signal 843 to the controller 100 to control the pressure in the air bladder of the garment 310 accordingly. The controller 100 may control the pressure according to the pressure value or multiple target pressure values in conjunction with the signal indicative of the measured pressure and/or the signal indicative of the determined movement.

Advantageously, by controlling the pressure in the air bladder of the garment 310 to change the level of intensity of the exercise being performed by the user, the settings and/or load and/or resistance of the exercise equipment the user is using to perform an example exercise movement do not need to be changed for the user to exercise at different intensities. For example, if the exercise movement to be performed is cycling, and the user is on an exercise bike, the controller 100 can change the level of intensity to mimic changing the resistance level of the exercise bike without actually doing so. Therefore the controller 100 can change the pressure of the air bladder of the garment 310 to mimic different workout types of an exercise bike, for example hill training, without requiring the resistance level of the exercise bike to be changed. Therefore the user can train at different intensities without their mechanical work rate changing. The mechanical work rate is defined as the rate at which a user moves a mechanical load, such as a particular weight or moves a piece of equipment which has resistance.

Another example exercise instruction indicates that an exercise movement is to be performed for a number of repetitions. When the exercise data 870 is retrieved by the electronic device processing means 810, the electronic device processing means 810 determines or is instructed that the exercise movement to be performed and the number of repetitions correspond to a target pressure value or multiple target pressure values for the garment 310 and sends a control signal 843 to the controller 100 to control the pressure in the air bladder of the garment 310 accordingly. The processing means 150 of the controller 100 and/or the electronic device processing means 810 determines when each repetition is completed by identifying the exercise movement by the user based on analysis of at least one of the signal of the measured pressure and the signal indicative of the determined movement.

Another example exercise instruction is to target maintaining the same mechanical work rate by the user for the period of time the exercise movement is to be performed by the user. For example, the user may be performing a bicep curl with a particular dumbbell weight during BFR training with the blood flow restriction garment 310 at an initial mechanical work rate. The processing means 150 and/or the electronic device processing means 810 can determine if the user is slowing down due to fatigue and therefore has a lower mechanical work rate, and in response to this the processing means 150 and/or the electronic device processing means 810 controls the valve 130 to decrease the pressure in air bladder, thereby reducing the effort required for the user to move the dumbbell and increasing the mechanical work rate back to the initial mechanical work rate.

The exercise data 870 and/or the exercise instructions can indicate a sequence of exercise movements to be performed sequentially by the user. When the exercise data 870 is retrieved by the electronic device processing means 810, the electronic device processing means 810 determines the target pressure values for the different exercise movements in the sequence and sends one or more corresponding control signals 843 to control the pressure in the air bladder accordingly for each exercise movement performed. The processing means 150 of the controller 100 and/or the electronic device processing means 810 determines or is instructed when each exercise movement is completed by identifying the exercise movement performed by the user based on analysis of at least one of the signal of the measured pressure and the signal indicative of the determined movement and then changes the pressure accordingly for the next exercise movement in the sequence.

In an example, the electronic device 210 is arranged to determine that the identified exercise movement deviates from the exercise data. For example, the electronic device processing means 810 retrieves the exercise data 870 and sends one or more control signals 843 to the controller 100 to control the pressure of the air bladder of the garment 310 accordingly. The electronic device processing means 810 can then determine or be instructed that the identified exercise movement performed by the user does not correspond to the exercise movement to be performed according to the exercise data 870.

The identified exercise movement could, for example, be recognised as a bad form version of the exercise movement to be performed according to the exercise data, or could be recognised as the user being stuck at part of the exercise movement. For example the exercise movement to be performed may be a squat, and the identified exercise movement may recognise that the user is not performing the full range of motion required or that they are stuck in a position midway through the squat due to fatigue. In other examples, the identified exercise movement may be a different exercise movement compared to the exercise movement to be performed according to the exercise data. For example, the exercise data may indicate a static squat is to be performed, but the identified exercise is a jumping squat.

In response to the electronic device 210 determining that the identified exercise movement deviates from the exercise data 870, the electronic device 210 may determine or be instructed that the pressure within the air bladder needs controlling to reduce the deviation. For example, if the user is stuck midway through a squat, the pressure in the air bladder of the garment 310 may be reduced to make it easier for the user to complete the squat, thereby completing the exercise movement to be performed so that the deviation between the identified exercise movement and the exercise movement to be performed according to the exercise data is reduced.

In response to the electronic device 210 determining that the identified exercise movement deviates from the exercise data 870, the electronic device 210 may determine or be instructed that the exercise data 870 needs changing in response to the deviation. For example, the exercise data 870 may initially include a jumping squat as an exercise movement to be performed. If the identified exercise movement is a static squat, it may be determined that the user would prefer to do static squats instead of jumping squats, and so the exercise data is changed so that the exercise movement to be performed is a static squat.

The exercise data 870 can be based on one or more data inputs. The one or more data inputs can be static data inputs, i.e. data that is fixed over a long period of time, and live data inputs from the controller 100 and/or the electronic device 210.

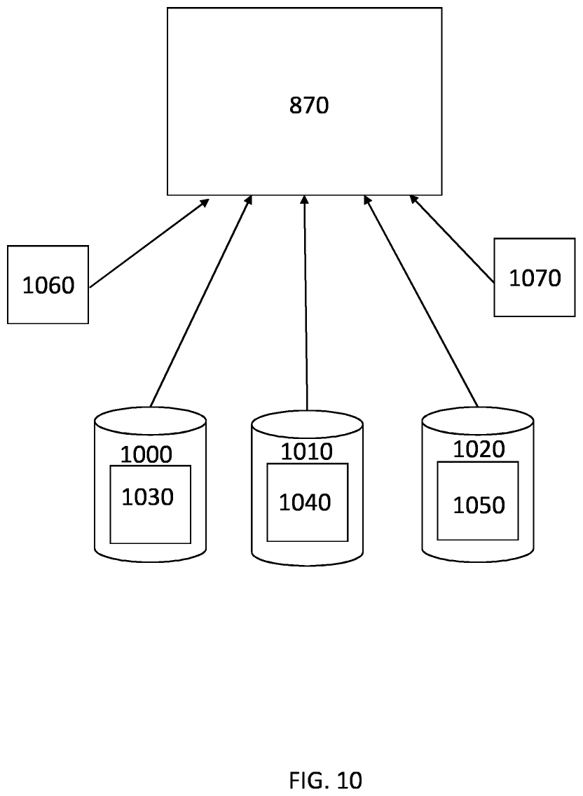

When the exercise data 870 is first generated, the static data inputs may be used. As illustrated in FIG. 10, these may be exercise movement data bank data 1030, expert data 1040, and user profile data 1050, stored in data stores 1000, 1010, 1020 in this example. The exercise movement data bank data 1030 comprises lists of different exercise movements that can be performed. The user profile data 1050 can comprise information about the user including age, sex, training frequency, level of function, pain levels, rate of perceived exertion (RPE), desired outcomes, etc., one or more of which may be entered in by the user, for example using the electronic device 210. The expert data 1040 can comprise percentage of limb occlusion pressure to be provided for the user, a recommended number of repetitions, sets, rest time, tempo, which can be based on the user profile data 1050 provided.

In some examples the data bank which comprises lists of different exercise movements to be performed is different from the data bank which is used to train the machine learning model 830. In some examples it is the same data bank.

The exercise data 870 can be generated from the static data using a recommender system. The recommender system may be performed by the electronic device 210 or another device which the electronic device 210 can communicate with. The recommender system may for example be a content-based filtering recommender system. The content-based filtering recommender system may first generate possible recommendations using the static data. The recommender system can then score the recommendations. The recommender system can then rank the recommendations and assign the highest ranked recommendations as the exercise data 870. Other recommender systems such as collaborative-based filtering recommender systems could also be used to generate and alter the exercise data.

After the user has started using the system 800, the exercise data may be updated in live time using additional data inputs, including data 1060 indicative of the measured pressure from the controller, and live metric data 1070, which can comprise data indicative of the determined movement, the identified exercise movement performed by the user, number of repetitions of the identified exercise movement performed by the user. The recommender system can then update the exercise data 870 using these additional data inputs.

The electronic device 210 in some examples can change the exercise data based upon expert data and the identified exercise movement by the user. In some examples the electronic device 210 can change the exercise data based upon the exercise movement data bank and the identified exercise movement by the user.

The electronic device 210 and/or the controller 100 are arranged to display information relevant to the exercise data on the screen 340 of the controller 100, and/or the display of the electronic device 210. For example the screen 340 and/or the display of the electronic device 210 may display a name of the exercise to be performed by the user according to the exercise data, a number of repetitions to complete, a number of repetitions completed, etc.

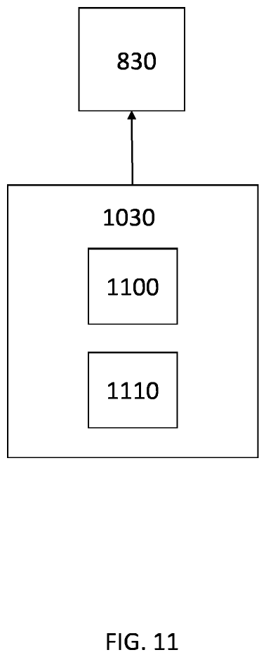

FIG. 11 illustrates a machine learning model 830 which is trained with exercise movement data bank 1030. The machine learning model may be trained to identify exercise movements using pressure data 1100 and movement data 1110 from the exercise movement data bank.

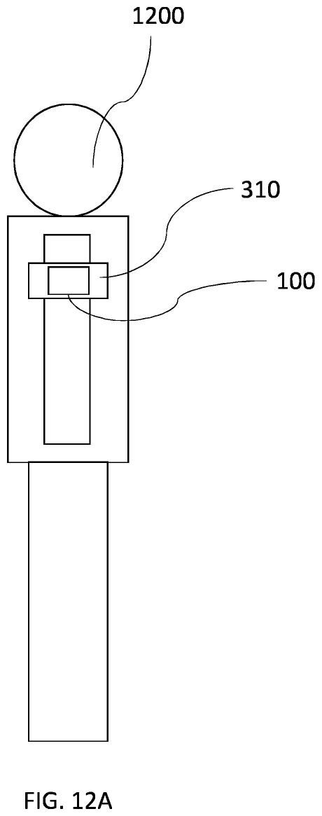

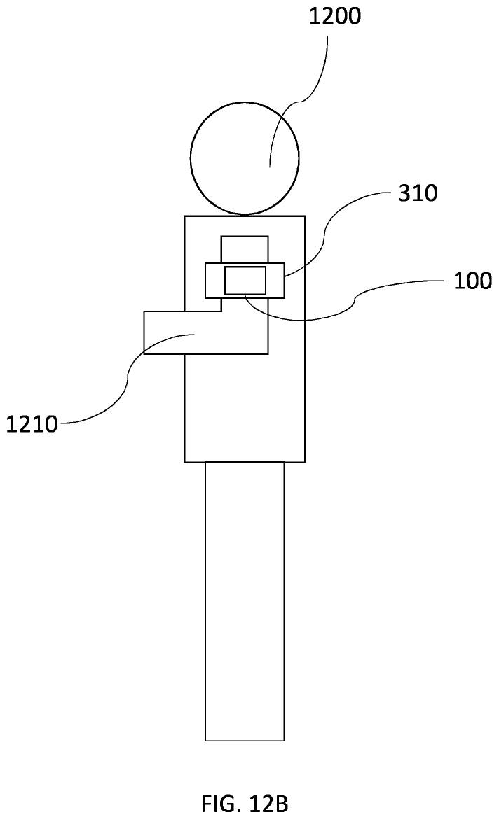

FIG. 12A illustrates a user 1200 wearing a blood flow restriction garment 310 and controller 100 attached to the blood flow restriction garment 310. In this example, the garment 310 is attached around the arm of the user 1200. The garment 310 may be designed to be attached around an arm and/or a leg of the user, or in other examples different garments may be used for the arm and for the leg, and the controller 100 can attach to either garment.

FIG. 12B illustrates the user performing an exercise movement 1210. In this example, the user is performing a bicep curl.

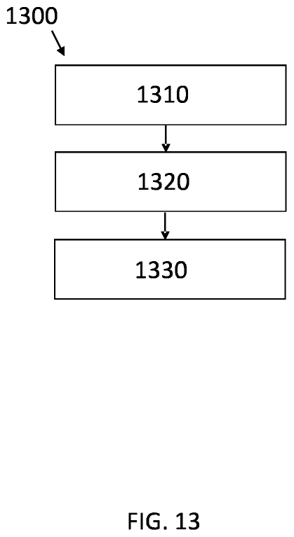

FIG. 13 illustrates an example method 1300 of controlling a blood flow restriction garment 310. The method 1300 comprises: receiving 1310, at a processing means 150 of a blood flow restriction garment controller 100, a signal indicative of a measured pressure of an air bladder of the blood flow restriction garment 310 from an air pressure sensor 120 of the blood flow restriction garment controller 100; receiving 1320, at the processing means 150 of the blood flow restriction garment controller 100, a signal indicative of a determined movement from at least one or more of an inertial measurement unit 140, an accelerometer 141, a gyroscope 142; providing 1330 a signal to control a valve 130 of the blood flow restriction garment controller 100 by selecting one of at least two operating states, the operating states comprising an open state and a closed state, wherein the signal to control the valve 130 is based on at least one of the signal indicative of the measured pressure and the signal indicative of the determined movement.

The method can comprise additional blocks according to features as described in previous examples.

The blocks illustrated in FIG. 13 may represent steps in a method 1300 and/or sections of code in a computer program configured to control the processing means as described above to perform the method steps. The illustration of a particular order to the blocks does not necessarily imply that there is a required or preferred order for the blocks and the order and arrangement of the block may be varied. Furthermore, it may be possible for some steps to be omitted or added in other examples.

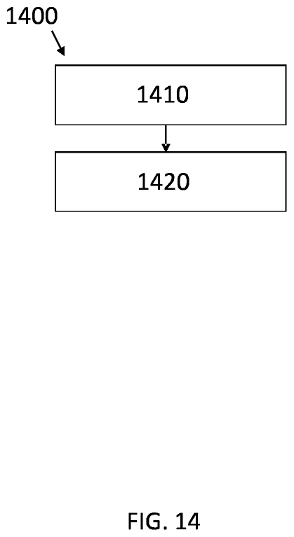

FIG. 14 illustrates an example method 1400 of controlling an electronic device 210. The method 1400 comprises: retrieving 1410 exercise data 870, using electronic device processing means 810 of the electronic device 210, wherein the exercise data 870 indicates at least one exercise movement to be performed by a user 1200 of a blood flow restriction garment 310; identifying 1420 exercise movement by the user 1200, using the electronic device processing means 810, based on analysis of at least one of a signal indicative of a measured pressure of an air bladder of the blood flow restriction garment 310 and a signal indicative of a determined movement using a machine learning model 830 trained with an exercise movement data bank 1030 comprising movement data and pressure data associated with different types of exercise movement; wherein the signal indicative of the measured pressure of the air bladder and the signal indicative of the determined movement are received from a blood flow restriction garment controller 100.

The method can comprise additional blocks according to features as described in previous examples. For example, the method can additionally comprise: controlling the pressure within the air bladder in dependence upon the identified exercise movement by the user 1200 and the exercise data; sending a control signal to a communication module 200 of the blood flow restriction garment controller 100 to control the valve 130 to control the pressure in the air bladder.

The blocks illustrated in FIG. 14 may represent steps in a method 1400 and/or sections of code in a computer program/software configured to control the processing means as described above to perform the method steps. The illustration of a particular order to the blocks does not necessarily imply that there is a required or preferred order for the blocks and the order and arrangement of the block may be varied. Furthermore, it may be possible for some steps to be omitted or added in other examples.

The examples illustrated above provide improved BFR training by providing a portable training system which can automatically change the pressure in the garment 310 to provide greater flexibility in training regimes. The controller 100 and system 800 can also improve the user's performance and/or recovery by determining how well the user is performing an exercise movement and helping them to correct their form or change the exercise movement or exercise conditions accordingly to help the user.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1-25. (canceled)

26. A blood flow restriction garment system, comprising:
a blood flow restriction garment controller;
an electronic device;
wherein the blood flow restriction garment controller comprises:
a pump arranged to increase a pressure in an air bladder of a blood flow restriction garment;
an air pressure sensor arranged to measure a pressure of the air bladder and provide a signal indicative of the measured pressure;
a valve arranged to control the pressure of the air bladder, wherein the valve has at least two operating states, wherein the operating states comprise an open state and a closed state;
at least one or more of an inertial measurement unit, an accelerometer, a gyroscope, arranged to determine movement of the blood flow restriction garment controller and provide a signal indicative of the determined movement;
processing means for providing a signal to control the valve by selecting one of the at least two operating states, wherein the signal to control the valve is based on at least one of the signal indicative of the measured pressure and the signal indicative of the determined movement;
a communication module for communicating with the electronic device;
wherein the electronic device comprises electronic device processing means,
wherein the electronic device processing means are arranged to:
retrieve exercise data, wherein the exercise data indicates at least one exercise movement to be performed by a user of the blood flow restriction garment;
identify an exercise movement by the user based on analysis of at least one of the signal indicative of the measured pressure and the signal indicative of the
determined movement using a machine learning model trained with an exercise movement data bank comprising movement data and pressure data associated with different types of exercise movement.

27. A blood flow restriction garment system as claimed in claim 26, wherein the blood flow restriction garment controller is arranged to transmit at least one of the signal indicative of the measured pressure and the signal indicative of the determined movement to the electronic device using the communication module.

28. A blood flow restriction garment system as claimed in claim 26, wherein the blood flow restriction garment controller comprises a housing arranged to removably attach to the blood flow restriction garment.

29. A blood flow restriction garment system as claimed in claim 28, wherein the controller comprises a connector for the air pressure sensor to connect to the air bladder and a connector for the pump to connect to the air bladder, wherein the housing comprises one or more apertures to enable the connector for the air pressure sensor and the connector for the pump to connect to the air bladder.

30. A blood flow restriction garment system as claimed in claim 26, wherein the blood flow restriction garment controller is arranged to be powered by portable energy storage means.

31. A blood flow restriction garment system as claimed in claim 26 arranged to maintain the pressure in the air bladder.

32. A blood flow restriction garment system as claimed in claim 26, wherein the processing means is arranged to control the pump in combination with controlling the valve to control the pressure in the air bladder.

33. A blood flow restriction garment system as claimed in claim 26 arranged to:
control the pressure within the air bladder in dependence upon the identified exercise movement by the user and the exercise data;
send a control signal to the communication module of the blood flow restriction garment controller to control the valve to control the pressure in the air bladder.

34. A blood flow restriction garment system as claimed in claim 26, wherein the exercise data indicates different levels of intensity for an exercise movement to be performed by the user, and wherein the electronic device determines that the pressure within the air bladder needs changing to enable the user to perform the exercise movement at the different levels of intensity.

35. A blood flow restriction garment system as claimed in claim 26, wherein the exercise data targets maintaining the same mechanical work rate by the user for a number of repetitions of an exercise movement to be performed by the user, and wherein the electronic device determines that the pressure within the air bladder needs controlling to maintain the same mechanical work rate by the user for the repetitions.

36. A blood flow restriction garment system as claimed in claim 26, wherein the exercise data targets maintaining the same mechanical work rate by the user for the period of time the exercise movement is to be performed by the user, and wherein the electronic device determines that the pressure within the air bladder needs controlling to maintain the same mechanical work rate by the user during the period of time.

37. A blood flow restriction garment system as claimed in claim 26, wherein the exercise data indicates a sequence of exercise movements to be performed sequentially by the user, and wherein the electronic device determines that the pressure within the air bladder needs controlling for the next exercise movement in the sequence.

38. A blood flow restriction garment system as claimed in claim 26, wherein the electronic device is arranged to determine that the identified exercise movement by the user deviates from the exercise data, and changes the exercise data in response to the deviation.

39. A blood flow restriction garment system as claimed in claim 26, wherein the electronic device is arranged to determine that the identified exercise movement by the user deviates from the exercise data, and determines that the pressure within the air bladder needs controlling to reduce the deviation.

40. A blood flow restriction garment system as claimed in claim 26, wherein the electronic device is arranged to change the exercise data based upon expert data and the identified exercise movement by the user and/or
wherein the electronic device is arranged to change the exercise data based upon the exercise movement data bank and the identified exercise movement by the user.

41. A blood flow restriction garment system as claimed in claim 26, wherein the exercise data is based initially on user data, expert data and the exercise movement data bank.

42. A blood flow restriction garment system as claimed in claim 28, comprising the blood flow restriction garment, wherein the blood flow restriction garment comprises a controller receiving portion arranged to receive the blood flow restriction garment controller to releasably attach the controller to the blood flow restriction garment.

43. A method of controlling an electronic device comprising:
retrieving exercise data, using electronic device processing means of the electronic device, wherein the exercise data indicates at least one exercise movement to be performed by a user of a blood flow restriction garment;
identifying exercise movement by the user, using the electronic device processing means, based on analysis of at least one of a signal indicative of a measured pressure of an air bladder of the blood flow restriction garment and a signal indicative of a determined movement using a machine learning model trained with an exercise movement data bank comprising movement data and pressure data associated with different types of exercise movement; wherein the signal indicative of the measured pressure of the air bladder and the signal indicative of the determined movement are received from a blood flow restriction garment controller.

44. A method as claimed in claim 43, additionally comprising:
controlling the pressure within the air bladder in dependence upon the identified exercise movement by the user and the exercise data;
sending a control signal to a communication module of the blood flow restriction garment controller to control the valve to control the pressure in the air bladder.

45. A computer program product which, which when executed on the electronic device processing means, is arranged to perform the method of claim 43.
